(19) United States
(12) Patent Application Publication
(10) Pub. No.: US 2014/0065226 A1
(43) Pub. Date: Mar. 6, 2014

(54) THERMO-RESPONSIVE HYDROGEL COMPOSITIONS

(71) Applicants: Eric Brey, Chicago, IL (US); Jennifer J. Kang-Mieler, Evanston, IL (US); Victor Perez-Luna, Naperville, IL (US); Bin Jiang, Chicago, IL (US); Pawel Drapala, Chicago, IL (US); Rolf Schäfer, Arisdorf (CH); Hans Hitz, Arisdorf (CH)

(72) Inventors: Eric Brey, Chicago, IL (US); Jennifer J. Kang-Mieler, Evanston, IL (US); Victor Perez-Luna, Naperville, IL (US); Bin Jiang, Chicago, IL (US); Pawel Drapala, Chicago, IL (US); Rolf Schäfer, Arisdorf (CH); Hans Hitz, Arisdorf (CH)

(21) Appl. No.: 13/261,732

(22) PCT Filed: Mar. 5, 2012
(86) PCT No.: PCT/EP2012/053765

§ 371 Date: Nov. 8, 2013

(30) Foreign Application Priority Data
Mar. 11, 2011 (US) 13/045643

(51) Int. Cl. A61K 47/32; A61K 31/573

(52) U.S. Cl. CPC A61K 47/32; A61K 31/573
USPC 424/490; 514/772.4; 514/179

(57) ABSTRACT

A thermo-responsive hydrogel, including a biocompatible monomer and/or polymer having a side chain-linked amino acid. The hydrogel is thermo-responsive at a physiological temperature, and can include, incorporate, or encapsulate a treatment agent, such as a drug composition, a biomolecule, and/or a nano-particle. The hydrogel is useful in delivering the treatment agent. The hydrogel is in a first physicochemical state for administration to a mammal. The hydrogel is thermo-responsive at a physiological temperature of the mammal, and changes to a second physicochemical state that is more solid than the first physicochemical state. In the second physicochemical state the thermo-responsive hydrogel releases the treatment agent.

### FIELD OF THE INVENTION

This invention relates generally to hydrogels and, more particularly, to hydrogels including biocompatible monomers, polymers and/or co-polymers comprising side chain-active, i.e., side chain-linked, amino acids, as well as to uses of these hydrogels, for medical treatments.

### BACKGROUND OF THE INVENTION

A hydrogel is a network of water-insoluble polymer chains that are hydrophilic. Hydrogels are suitable for various biomedical applications, such as tissue treatment and delivery mechanisms. Their high water content and the fact that they can be formed under mild reaction conditions makes them attractive for applications involving encapsulation of cells and labile biomolecules such as proteins. Cross-linked hydrogels are capable of encapsulating biomaterials, which are then protected by a semi-permeable hydrogel barrier that prevents immune system attack or degradation by proteases.

Thermo-responsive hydrogels are ideally suited for localized delivery applications with minimum invasiveness due to a change in physicochemical properties in response to temperature. Thermo-responsive hydrogels can be administered as liquid-like gels that, upon reaching body temperature, solidify at the site of injection. Thermo-responsive hydrogels may be synthesized using natural polymers such as the polysaccharides chitosan, dextran and cellulose or using proteins such as gelatin. Hydrogels based on poly(N-isopropylacrylamide) have attracted interest due to a sharp lower critical solution temperature behavior around 32° C., which can be suitable for biomedical applications. For cross-linked materials, this thermal transition temperature is often referred to as volume phase transition temperature.

Achieving the desired release kinetics with poly(N-isopropylacrylamide) hydrogels can pose specific challenges. Furthermore, poly(N-isopropylacrylamide) hydrogel materials have relatively low cell and tissue adhesive properties which are important in wound healing. There is thus a need for improved hydrogel materials, particularly for use in medical treatments and/or as treatment delivery systems.

### SUMMARY OF THE INVENTION

A general object of the invention is to provide a hydrogel composition, also referred to herein as a “thermo-responsive hydrogel composition,” a “thermo-responsive hydrogel,” or simply a “hydrogel,” having improved biological properties, such as having desirable release kinetics and/or cell and tissue adhesion.

The general object of the invention can be attained, at least in part, through a thermo-responsive hydrogel including a biocompatible monomer and/or polymer having an amino acid side chain (i.e., having an amino acid linked to the remainder of the monomer or polymer through its side chain). The hydrogel is desirably thermo-responsive at a physiological temperature, and can include, incorporate, and/or encapsulate a treatment agent, such as a drug composition, a biomolecule, and/or a nanoparticle.

Optionally, in any embodiment of the invention, the biocompatible monomer or polymer comprises an amino acid linked through its side chain to an acrylic-, maleinic-, or phtalic-derivative. Optionally, in any embodiment of the invention, the amino acid is lysine, tyrosine, serine, cysteine, proline, or combinations or derivatives thereof. Optionally, in any embodiment of the invention, the thermo-responsive hydrogel comprises a hydrophilic polymer reacted with a crosslinker. Optionally, the crosslinker comprises poly(ethylene glycol)diacrylate, bisacrylamide, or dithiol functionalized molecules. Optionally, in any embodiment of the invention, the thermo-responsive hydrogel comprises N-isopropylacrylamide. Optionally, in any embodiment of the invention, the N-isopropylacrylamide is crosslinked with poly(ethylene glycol)diacrylate, bisacrylamide, or dithiol functionalized molecules. In one embodiment, the thermo-responsive hydrogel comprises N-isopropylacrylamide and the biocompatible monomer or polymer comprises lysine, tyrosine, serine, cysteine, proline, or combinations or derivatives thereof. Optionally, in any embodiment of the invention, a composition further comprises N-tert-butylacrylamide (NtBAAm). Optionally, in any embodiment of the invention, a composition is a wound treatment. Optionally, in any embodiment of the invention, a composition further comprises an antimicrobial agent. Optionally, in any embodiment of the invention, a composition is a topical ocular treatment. Optionally, in any embodiment of the invention, a composition further comprises an encapsulated drug composition. Optionally, the encapsulated drug composition is encapsulated in a nanosphere. Optionally, the encapsulated drug composition comprises nanosphere encapsulated dexamethasone.

In one embodiment, the invention provides a hydrogel composition, comprising: a thermo-responsive crosslinked acrylamide polymer; a biocompatible monomer or polymer including a side chain-linked amino acid; and a drug composition, a biomolecule, and/or a nanoparticle. Optionally, the thermo-responsive crosslinked polymer comprises N-isopropylacrylamide crosslinked with poly(ethylene glycol)diacrylate. Optionally, the amino acid is lysine, tyrosine, serine, cysteine, proline, or combinations or derivatives thereof.

Optionally, in any embodiment of the invention, the biocompatible monomer or polymer comprises l-, d- or d,l-amino acids linked through their side chains to acrylic-, maleinic-, or phtalic-derivatives. Optionally, in any embodiment of the invention, the composition is a wound treatment, and the drug composition comprises an antimicrobial agent. Optionally, in any embodiment of the invention, composition is a topical ocular treatment, and the drug composition is encapsulated in a nanosphere.

The invention further comprehends a method of delivering a treatment agent. The method includes providing a thermo-responsive hydrogel including the treatment agent, wherein the hydrogel is thermo-responsive at a physiological temperature; administering to a mammal the thermo-responsive hydrogel in a first physicochemical state; and the thermo-responsive hydrogel changing to a second physicochemical state upon administration, wherein the second physicochemical state is more solid than the first physicochemical state. In the second physicochemical state the thermo-responsive hydrogel releases the treatment agent.

The invention also encompasses use of the compositions for the invention in medical treatments and/or as treatment delivery systems (e.g. for treatment of a wound, an ocular disease or condition, etc).

Other objects and advantages will be apparent to those skilled in the art from the following detailed description taken in conjunction with the appended claims and drawings.

### BRIEF DESCRIPTION OF THE DRAWINGS

FIG. 1 is an image of a vial including a cross-linked thermo-responsive hydrogel at room temperature.

FIG. 2 is an image of a vial including a cross-linked thermo-responsive hydrogel at 37° C.

FIG. 3 is a bright-field image of the cross-linked thermo-responsive hydrogel of FIG. 1.

FIG. 4 is a bright-field image of a gel edge of the cross-linked thermo-responsive hydrogel of FIG. 2.

FIG. 5 is a graph of the lower critical solution temperature of PNIPAAm alone and cross-linked PNIPAAm-PEG-DA hydrogels.

FIG. 6 illustrates a reaction scheme forming cross-linked, thermo-responsive PEG-DA/NIPAAm hydrogel with encapsulated BSA.

FIG. 7 is a graph of a surface infection evaluation from a swab test. * indicates p<0.05. At each time (postsurgical), the leftmost bar represents Control, the middle bar represents Hydrogen with PBS and the rightmost bar represents Hydrogen with 0.1% PHMB+0.5% CHX.

FIG. 8 is a graph of a deep infection evaluation at days 4, 8 and 12. The line indicates threshold bacteria count for wound infection. At each time (postsurgical), the leftmost bar represents Control, the middle bar represents Hydrogen with PBS and the rightmost bar represents Hydrogen with 0.1% PHMB+0.5% CHX.

FIG. 9 shows in vitro activity of DSP release from pure hydrogel, free nanospheres, and nanospheres loaded hydrogel. PBS was used as control. At each time point, the leftmost bar represents Control, the bar second from the left represents Pure hydrogel, the bar third from the left represents Free nanospheres, and the rightmost bar represents Nanospheres loaded hydrogel.

FIG. 10 includes images of a progression of uveitis for DSP-nanosphere-hydrogels: A) IR image prior to LPS injection, B) IR images 24 hrs post injection, C) IR image 48 hrs post injection/24 hrs post treatment, D) IR image 72 hrs post injection/48 hrs post treatment, and E) IR image 96 hrs post injection/72 hrs post treatment.

FIG. 11 includes images of a progression of uveitis for DSP solution: A) IR image prior to LPS injection, B) IR images 24 hrs post injection, C) IR image 48 hrs post injection/24 hrs post treatment, D) IR image 72 hrs post injection/48 hrs post treatment, and E) IR image 96 hrs post injection/72 hrs post treatment.

FIG. 12 includes images of a progression of uveitis for no treatment: A) IR image prior to LPS injection, B) IR images 24 hrs post injection, C) IR image 48 hrs post injection, D) IR image 72 hrs post injection, and E) IR image 96 hrs post injection.

FIG. 13 includes anterior images of the eye of FIG. 10 treated with DSP-nanosphere loaded hydrogel: A) prior to LPS injection, B) 24 hrs post injection, C) 48 hrs post injection/24 hrs post treatment, D) 72 hrs post injection/48 hrs post treatment, and E) 96 hrs post injection/72 hrs post treatment.

FIG. 14 includes is anterior images of the eye of FIG. 11 treated with DSP solution: A) prior to LPS injection, B) 24 hrs post injection, C) 48 hrs post injection/24 hrs post treatment, D) 72 hrs post injection/48 hrs post treatment, and E) 96 hrs post injection/72 hrs post treatment.

FIG. 15 includes anterior images of the eye of FIG. 12 receiving no treatment: A) prior to LPS injection, B) 24 hrs post injection, C) 48 hrs post injection, D) 72 hrs post injection, and E) 96 hrs post injection.

FIG. 16 summarizes an evaluation of inflammation with Treatment 1 of the Examples based on a grading system (scale: 0-4).

FIG. 17 summarizes an evaluation of inflammation with Treatment 2 of the Examples based on a grading system (scale: 0-4).

FIG. 18 summarizes an evaluation of inflammation with no treatment based on a grading system (scale: 0-4). * indicates p<0.05.

FIG. 19 illustrates fluorescence readings of a Pico Green assay. Blank: PBS buffer; Background: hydrogels with no cells; 0%-5% A-lysine: hydrogels with corresponding A-lysine concentration and seeded with cells.

FIG. 20 summarizes dexamethasone release from prepared nanospheres.

FIG. 21 summarizes dexamethasone sodium phosphate release from nanospheres and microspheres.

FIG. 22 includes control LPS model images: A) a SLO image before the LPS injection; B) 24 hours after the injection of control hydrogel (no drug); and C) day 6 after the LPS injection.

FIG. 23 includes images of comparisons of treatment method: A) control image prior to the LPS injection; B) day 2 (24 hr after the dexamethasone treatment); C) day 6 with the dexamethasone treatment); D) control image prior to the LPS injection; E) day 2 (24 hr after the dexamethasone hydrogel treatment); and F) day 6 image with dexamethasone hydrogel treatment.

FIG. 24 includes images of subconjunctival injection of thermo-responsive hydrogel: A) control image before the LPS injection; B) day 2 after the subconjunctival injection; and C) day 6.

### DETAILED DESCRIPTION OF THE INVENTION

The present invention provides compositions including a thermo-responsive hydrogel and a biocompatible monomer or polymer including an amino acid side chain (i.e., having an amino acid linked to the remainder of the monomer or polymer through its side chain). In one embodiment of this invention, the hydrogel is thermo-responsive at a physiological temperature, such that a physicochemical change occurs around a typical body temperature, such as at or above about 32° C., and generally from about 32° C. to about 39° C., and more preferably from about 32° C. to about 37° C. The compositions of this invention having thermo-responsive behavior at physiological temperature are useful as injectable and topical formulations, particularly for biomedical applications such as, without limitation, localized drug delivery, wound treatments and coverings, tissue engineering, dental applications, cartilage regeneration, bulking agents for incontinence treatments, and tissue fillers in reconstructive and cosmetic surgery.

In one embodiment of this invention, the thermo-responsive hydrogels have fluid-like consistency at room temperature and transform into a viscoelastic solid upon reaching physiological temperatures. The thermo-responsive hydrogels are formed by crosslinking a monomer and/or polymer, and desirably a hydrophilic monomer or polymer. Any suitable materials can be used to form the thermo-responsive hydrogels of this invention. Exemplary thermo-responsive hydrogels of this invention are formed of one or more crosslinked acrylamide polymers. A preferred acrylamide is N-isopropylacrylamide (NIPAAm), used to form poly(N-isopropylacrylamide) (PNIPAAm) hydrogel. A suitable method for synthesis of hydrogels is through free radical copolymerization with crosslinkers such as N,N-methylenebis-acrylamide (MBIS), poly(ethylene glycol)diacrylate (PEG-DA), bisacrylamide, dithiol functionalized molecules that can crosslink through Michael's addition reaction, and/or other covalent, ionic, hydrophobic interactions. Crosslinking can also occur through condensation, free radicals, reduction and oxidation reactions that produce crosslinking of hydrogel precursors. Other suitable, but often more complicated methods to crosslink PNIPAAm involve formation of interpenetrating polymer networks, polysaccharides such as dextran, or multi-arm PEG-DA cross-linkers.

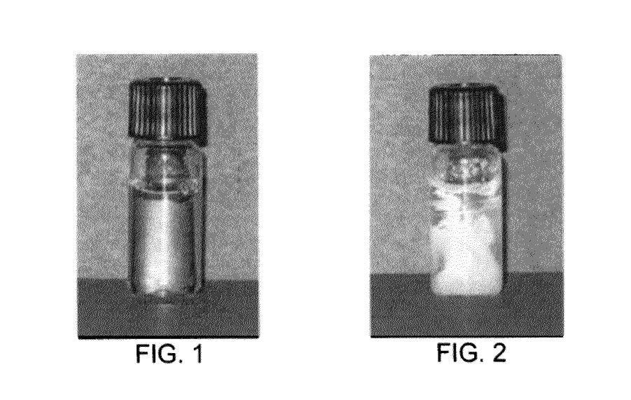

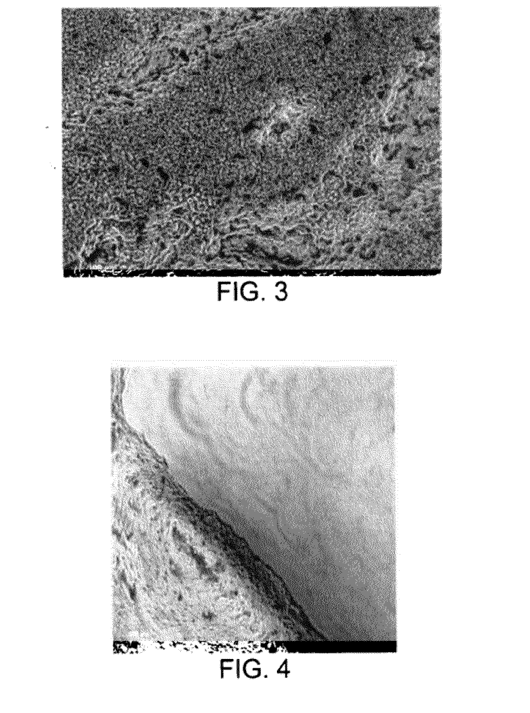

The PNIPAAm-PEG-DA hydrogel is a particularly desirable polymer, having unique biocompatibility and polymerization characteristics. PNIPAAm-PEG-DA hydrogel is soluble in water and is readily cleared by the body. PNIPAAm-PEG-DA hydrogel can be immobilized either chemically or physically, is highly resistant to protein adsorption and cell adhesion, and is not readily recognized by the immune system. Acrylates are used as end groups because they undergo very rapid photopolymerization. By incorporating PEG-DA with PNIPAAm in the polymerization process, a nondegradable formulation is achieved. FIGS. 1 and 2 show sample images of the PNIPAAm-PEG-DA hydrogel at room temperature and at a physiological temperature of 37° C. At room temperature (˜20° C.), shown in FIG. 1, the hydrogel existed in a liquid gel-like phase. Raising the temperature to 37° C. caused the hydrogel to rapidly form a solid gel rapidly (within 1 minute). Bright-field images of the gel surfaces and edges in FIGS. 3 and 4 show a relatively uniform pore surface created by the cross-linking process.

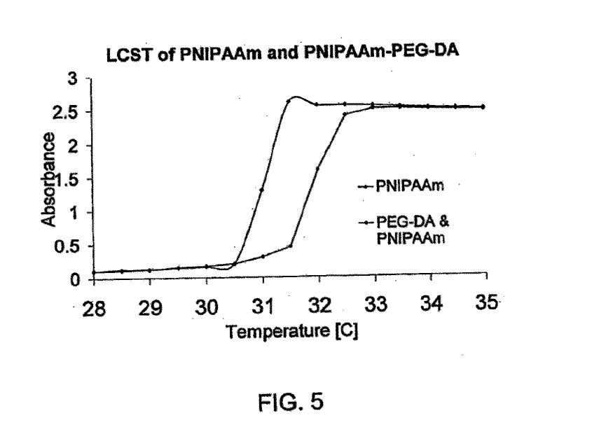

The lower critical solution temperature (LCST) of PNIPAAm alone and cross-linked PNIPAAm-PEG-DA, obtained by measuring the average absorbance of the hydrogel as a function of temperature, is shown in FIG. 5 (PNIPAAm alone is the line having the lower temperature at an absorbance of 1 and PNIPAAm-PEG-DA is the line having higher temperature at an absorbance of 1). A hydrogel of PNIPAAm alone changed its phase (LCST) at ˜31° C. PNIPAAm-PEG-DA hydrogel changed its phase at ˜32° C. By cross-linking with PEG-DA, LCST was shifted by ˜1° C., likely owing to the increased hydrophilicity, but still within an optimal range of injection.

As discussed above, the physicochemical change of the thermo-responsive hydrogel materials that occurs at physiological temperature is particularly useful for biomedical applications. However, these hydrogel materials typically have low cell and tissue adhesive properties. It has been found that the introduction of biocompatible monomers and/or polymers including an amino acid side chain, i.e., a side chain-linked amino acid, into the hydrogel materials enhances cellular interactions of the hydrogels, while still maintaining the thermo-responsive characteristics. Suitable biocompatible monomers and/or polymers for use in the thermo-responsive hydrogel compositions of this invention are disclosed in International Patent Application PCT/IB2006/1001722 (WO 2006/126095), herein incorporated by reference.

In one embodiment of this invention, the biocompatible monomer or polymer comprises an amino acid linked through its side chain but not its alpha-aminocarboxy functionality to an acrylic-, maleinic-, or phtalic-derivative. Suitable and desirably amino acids include lysine, tyrosine, serine, cysteine, proline, or combinations or derivatives thereof. Exemplary biocompatible monomers include bifunctional l-, d- or d,l-amino acids side chain-linked to acrylic-, maleinic-, or phtalic-derivatives. Exemplary biocompatible monomers include, without limitation: acryloyl-lysine; acryloyl-tyrosine; acryloyl-serine; acryloyl-cysteine; acryloyl-proline; methacryloyl-lysine; methacryloyl-tyrosine; methacryloyl-serine; methacryloyl-cysteine; methacryloyl-proline; maleicacid-, 2-methylmaleicacid-, 2,3-dimethylmaleicacid-, or phtalicacid-N-lysine-amide; maleicacid-, 2-methylmaleicacid-, 2,3-dimethylmaleicacid-, or phtalicacid-O-tyrosine-ester; maleicacid-, 2-methylmaleicacid-, 2,3-dimethylmaleicacid-, or phtalicacid-O-serine-ester; maleicacid-, 2-methylmaleicacid-, 2,3-dimethylmaleicacid-, or phtalicacid-S-cysteine-thioester; and/or maleicacid-, 2-methylmaleicacid-, 2,3-dimethylmaleicacid-, or phtalicacid-O-proline-ester.

In one embodiment of this invention, the LCST of the thermo-responsive hydrogel composition can be altered or “tuned” higher or lower, depending on need. The LCST can be altered by incorporating N-tert-butylacrylamide (NtBAAm), chain transfer agents, and/or monomers that can affect the hydrophilic/hydrophobic character of the hydrogel. In another embodiment of this invention, altering the LCST of the hydrogel degradation products can be done to provide degradation products that are soluble at physiological temperatures, thereby facilitating clearance from the body upon hydrogel degradation.

The thermo-responsive hydrogel compositions of this invention can encapsulate or otherwise contain or incorporate a treatment agent. As used herein, “treatment agent” refers to any material or composition to be delivered onto or into a body. Exemplary treatment agents include drug compositions, biomolecules, and/or nanoparticles. Various and alternative drug compositions, biomolecules, and nanoparticles are available for use with the hydrogel compositions, depending on need. The drug composition can be an antimicrobial such as Cosmocil CQ-20% polyhexamethylene biguanide (PHMB), or other known drug compositions such as, without limitation: prodrugs; antibiotics such as aminoglycosides (gentamicin, neomycin, and tobramycin), macrolides (erythromycin), fluoroquinolones (ciprofloxacin, levofloxacin, ofloxacin, gatifloxacin, and moxifloxacin), and others including chloramphenicol and natamycin; steroids and anti-inflammatory molecules and agents, such as dexamethasone, dexamethasone sodium phosphate (DSP), fluorometholone, and prednisolone acetate; growth factors; endocrine and paracrine signals; and anti-VEGF agents such as bevacizumab, ranibizumab, pegaptanib, and VEGF-trap. The drug compositions can be encapsulated in nanoparticles or nanospheres, such as poly(lactide-co-glycolide) (PLGA) nanospheres/nanoparticles. Additional drug delivery carrier systems can be incorporated within these hydrogels, such as, but not limited to, lyposomes, polymersomes, nanoparticles, micellar systems, dendrimers, bioactive polymers, and prodrug crystals. Exemplary biomolecules include proteins, enzymes, enzyme inhibitors, DNA, RNA, endocrine and paracrine signals, and/or therapeutic cells or factors, such as for the treatment of tissue (limb/myocardial) ischemia.

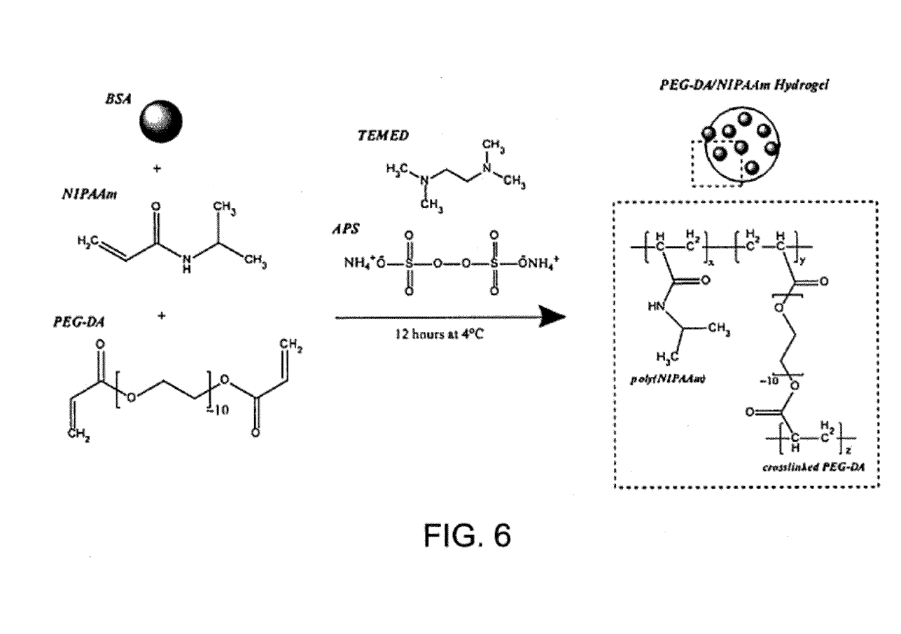

Treatment agents such as drug compositions, biomolecules, and/or nanoparticles can be loaded into the hydrogel compositions either at room temperature or at temperatures lower than the LCST of the hydrogel, and prior to polymerization or after. FIG. 6 illustrates an exemplary reaction scheme for encapsulating the protein bovine serum albumin (BSA). The reaction in FIG. 6 started with a 25 mg/ml BSA solution in degassed PBS (pH 7.4). The hydrogel compositions can be prepared by dissolving PEG-DA (concentrations of 4, 8, 12 or 16 mM according to the desired hydrogel consistency) either in the BSA solution or PBS. Cross-linking concentrations can be selected based on the ability for the hydrogel to be injected via a small hypodermic needle (i.e., the hydrogels had to have fluid-like consistency). The hydrogels typically become too rigid for injection using small gauge needles at above 16 mM PEG-DA. Below 4 mM PEG-DA the hydrogels can be difficult to manipulate because of the low cross-linked nature. The monomer NIPAAm is dissolved in the solution to a final concentration of 0.35 M. After complete dissolution of NIPAAm, polymerization of the hydrogel is initiated by adding ammonium persulfate (APS) (e.g., 13 mM) and N,N,N′,N′-tetramethylethylenediamine (TEMED) (e.g., 168 mM). This procedure results in free radical polymerization, which is allowed to proceed at 4° C. for 12 hours to result in the formation of cross-linked thermo-responsive hydrogel compositions containing encapsulated BSA.

Following the 12 hour polymerization period, unreacted monomer and initiators are desirably removed by extraction through gentle agitation of the hydrogels in PBS buffer for 20 min. As initiators and unreacted monomers can cause toxicity, extraction of these components is essential for minimizing cell toxicity. Five washes have been shown to sufficiently remove residuals, resulting in a material that does not exhibit cell toxicity. Hydrogel compositions of this invention can also be sterilized with ethylene oxide gas and maintain the thermo-responsive behavior. Precursors can be maintained sterile prior to hydrogel formation and hydrogel synthesis can proceed in sterile environments.

The invention also includes the use of a thermo-responsive hydrogel in a method of delivering a treatment agent. The desired treatment agent, such as a drug composition, biomolecule, and/or nanoparticle is incorporated or embedded within the hydrogel. The hydrogel is desirably administered in a first physicochemical state for application, such as by topical application or by local, systemic, transdermal, or transcorneal injections. Ocular treatments, for example, both with and without nanospheres, can be delivered topically such as by eye drops or inserts under the eyelids, subtenon injections, subconjunctival injections, and/or intravitreal injections. Upon administration to a mammal, the hydrogel encounters a physiological temperature that causes the thermo-responsive hydrogel to change to a second physicochemical state upon administration. The second physicochemical state is more solid than the first physicochemical state, such as discussed above and illustrated in FIGS. 1-4. In the second physicochemical state, the hydrogel releases the treatment agent. In one embodiment, the release occurs as the hydrogel degraded.

The physiochemical change to a more solid state upon reaching body temperatures provides for efficient application of a more fluid gel-like material that then solidifies after application or administration. As discussed above, the hydrogel compositions of this invention are particularly useful in ocular applications, such as for delivering anti-inflammatory agents, such as encapsulated dexamethasone or dexamethasone sodium phosphate (DSP), ocular tumor treatments, anti-VEGF agents, and/or other drugs such as antibiotics, growth factors, steroids, enzymes, enzyme inhibitors. The hydrogel compositions of this invention are also particularly useful in wound coverings and/or skin regeneration. A topical hydrogel wound covering can be applied and changed as needed, such as daily or weekly, etc. The topical hydrogel can include treatment agents such as antibiotics and/or regeneration drugs and/or biomaterials. Other uses include, without limitations: delivery of therapeutic cells or factors for the treatment of tissue (limb/myocardial) ischemia; delivery of anti-angiogenic drugs for tumor treatment; providing a scaffold for tissue engineering applications; dental applications, such as for extracted teeth; antimicrobial and regeneration cartilage regeneration; bulking agents for incontinence treatments; and tissue fillers in reconstructive and cosmetic surgery.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

The following examples demonstrated that a thermo-responsive hydrogel can be incorporated with acryloyl-lysine (A-lysine) according to this invention, while maintaining the thermo-responsive characteristics.

### Example 1

### Wound and Ocular Applications

### Materials

Poly(lactide-co-glycolide) 50:50 (PLGA 50:50; ave. Mw 7,000-17,000, ester terminated), polyvinyl alcohol (PVA; ave. Mw 30,000-70,000), poly(ethylene glycol)diacrylate (PEG-DA; ave. Mn=575), N-isopropylacrylamide (NIPAAm) 97%, n-tert-butylacrylamide (NtBAAm) 97%, N,N,N′,N′-tetramethylethylenediamine (TEMED), Ammonium persulfate (APS), Lipopolysaccharides (LPS) from Salmonella Typhimurium and chlorehexidine digluconate solution (20% in water) were obtained from Sigma-Aldrich. Dichloromethane and methanol were obtained from Fisher Scientific in HPLC grade. Difco™ nutrient broth and Bacto™ agar were purchased from BD Biosciences. A-lysine (N6-acryloyl lysine) was provided by CIS Pharma (Bubendorf, Switzerland). Cosmocil CQ (20% polyhexamethylene biguanide solution) was obtained from Organic Creations. Dexamethasone 21-phosphate disodium salt ≧98% was obtained from MP Biomedicals.

### Hydrogel Preparation for Dermal Application

Poly (NIPAAm)-PEG hydrogels with A-lysine and NtBAAm were synthesized by free radical polymerization using 3 mg/ml APS as an initiator and 30 μl/ml TEMED as an accelerator in an ice bath for an hour. Specifically, hydrogels with 5% A-Lysine and 15% NtBAAm (w/w NIPAAm) were synthesized for ocular application; hydrogels with 5% A-lysine and 20% NtBAAm (w/w NIPAAm) were synthesized for dermal application. After the hydrogel synthesis, unreacted monomers and initiators were extracted by washing in PBS for 5 times, with changing of fresh PBS every 20 minutes.

Polyhexamethylene biguanide (PHMB) 0.1% and chlorhexidine digluconate 0.5% (w/v) in PBS solution was loaded into the hydrogel by equilibrating the mixed drug solution overnight for dermal application. After hydrogel synthesis and drug loading, the hydrogels were kept at 4° C. for storage. All hydrogels were prepared under sterile conditions.

### Wound Infection Animal Experiment

Pseudomonas aeruginosa (ATCC #19660) were cultured overnight in 0.8% Difco™ nutrient broth media at 37° C. with constant shaking at 275 rpm. Subcultures were transferred to a 50 ml tube and centrifuged at 4000 rpm for 10 min at 4° C. Resulting bacterial pellets were washed twice and resuspended in PBS, and placed on ice prior to inoculation. The bacterial concentration of 100 μl sample was first estimated spectrophotometrically at wavelength 620 nm using the formula concentration (cfu/ml)=OD620×2.5×10⁸. The bacterial concentration was then verified by serial dilution on 1% Bacto™ agar plates with 0.8% Difco™ nutrient broth media, and colony counting after overnight culture at 37° C. with ambient air.

Adult male Sprague-Dawley rats (weight 200˜250 g) were used. The animals were anesthetized, shaved, disinfected, and an 8-mm punch biopsy tool was used to create two circular, full-thickness cutaneous wounds on the middle of shaved dorsal skin. A donut shaped silicone splint was centered on the wound and affixed using cyanoacrylate adhesive and interrupted 4-0 nylon sutures. Inoculation of bacteria was performed immediately after the animal surgery. The bacteria were diluted to 10⁹ CFU/ml in sterile PBS and 100 μl of bacteria suspension was added using a micropipette to each wound bed. A semiocclusive dressing (Tegaderm; 3M) was applied double layered to cover the wound after the inoculation. One day after the surgery and inoculation, 200 μl of hydrogel loaded with 0.1% PHMB and 0.5% chlorhexidine digluconate was applied to individual wound sites.

### Wound Infection Analysis

The animals (9 rats) were anesthetized at day 4, 8, and 12 after the surgery and a swab test culture was used to evaluate infection. A cotton-tipped swab from the BD E-Swab kit was used to sample the superficial wound fluid and tissue debris. The sample was then transferred to an appropriate diluent using the BD E-Swab collection kit. The suspensions were then serial diluted from 1:10³ to 1:10¹² with sterile broth media and the dilutions were plated on broth-agar plates to quantify bacteria concentration. The animals were sacrificed with CO₂ inhalation after the swab test, and the skin including the entire wound with adjacent normal skin was excised as a 2.5 cm×2.5 cm square. Each harvested skin square tissue was divided into two. One half was fixed in 10% formaldehyde buffered solution for histological analysis and the other placed on ice for deep skin infection analysis. The tissue sample for infection analysis was weighed and homogenized using a sterile mortar and pestle, after which the homogenized tissue was suspended in 2 ml sterile PBS. Suspensions were serial diluted from 1:10³ to 1:10¹² with sterile broth media and plated on broth-agar plates at 37° C. for 24 h in ambient air. Bacterial counts were expressed as numbers of bacterial colony forming units per gram (cfu/g) of tissue. Typically, >10⁵ cfu/g is considered infected.

### Nanosphere-Loaded Hydrogel Preparation for Ocular Application

PLGA (50 mg) was dissolved in dichloromethane (1 ml) followed by the addition of 0.1 ml DSP methanol solution (50 mg/ml). The clear organic mixture was emulsified into an external aqueous phase (5 ml, 2% w/v PVA) with vortexing for 20 seconds followed by sonication on ice at 55 W for 5 minutes. The resultant emulsion was stirred at 250 rpm for over 3.5 hours to allow organic solvent evaporation and nanosphere precipitation. Nanospheres were harvested by ultracentrifugation at 16,000 g for 10 min, after which the resultant pellet was re-suspended in DI water by sonication and washed twice with DI water. To quantify drug encapsulation, the PLGA nanospheres were incubated overnight in 1 N NaOH solution at 37° C. to allow complete PLGA degradation. The resultant solution was read spectrophotometrically at 240 nm for DSP concentration. Dynamic light scattering (DLS) was used to characterize the size of the nanospheres. To investigate nanosphere encapsulation in thermo-responsive hydrogels, nanospheres were added into the hydrogel precursor solutions at 0, 2.5, 5, and 10 mg/ml prior to polymerization initiated by the addition of TEMED. The hydrogels were then washed 5 times with PBS every 20 minutes, as described for hydrogels with no nanospheres. Swelling ratio was tested for hydrogels with varying concentration of nanospheres at both room and body temperature.

In Vitro Activity of DSP Release from Nanospheres Loaded Hydrogel

PLGA nanospheres with DSP were loaded into the thermo-responsive hydrogels at 10 mg/ml. Drug release was carried out in PBS at 37° C. As a comparison, DSP loaded directly into the hydrogel and free nanospheres were also placed in PBS for drug release at 37° C. Drug release samples were taken at predetermined time intervals and tested for anti-proliferative activity with fibroblast MTS assay as described previously. Briefly, 3T3 fibroblast cells were seeded in 96-well plates as 5000 cells/well. After cells were grown to semiconfluence, growth was arrested by washing plates with PBS and then adding low serum medium with DMEM, 0.5% (v/v) FBS and 1% (v/v) penicillin/streptomycin mixture. Growth arrest was maintained for 24 hours. The cell cycle synchronized cells were then re-stimulated to enter G1 phase by changing back to growth medium. DSP release samples from different delivery system were added to cells at the time of serum re-stimulation. PBS was added to growth medium as control group. MTS assay was used to determine cell proliferation after 2 days of DSP exposure.

### Endotoxin-Induced Uveitis In Vivo Experiment

Adult male Lewis rats (weight ˜175-250 grams) were used in endotoxin-induced uveitis (EIU) animal model. LPS from Salmonella Typhimurium (Sigma Aldrich; St. Louis, Mo.) was mixed with PBS immediately prior to injection. The animal's eyes were treated with various treatment regimens as described below:

Treatment 1: intravitreal injection of LPS at 0 hours. ˜20 μl of DSP encapsulated nanosphere and hydrogel (4 mg/ml) placed under the eyelids 24 hours post LPS and daily for up to 96 hours;

Treatment 2: intravitreal injection of LPS at 0 hours. ˜20 μl of DSP solution (4 mg/ml) placed directly on the cornea (simulate eyedrops) 24 hours post LPS and daily up to 96 hours; and

No Treatment: intravitreal injection of LPS at 0 hours and follow up daily up to 96 hours.

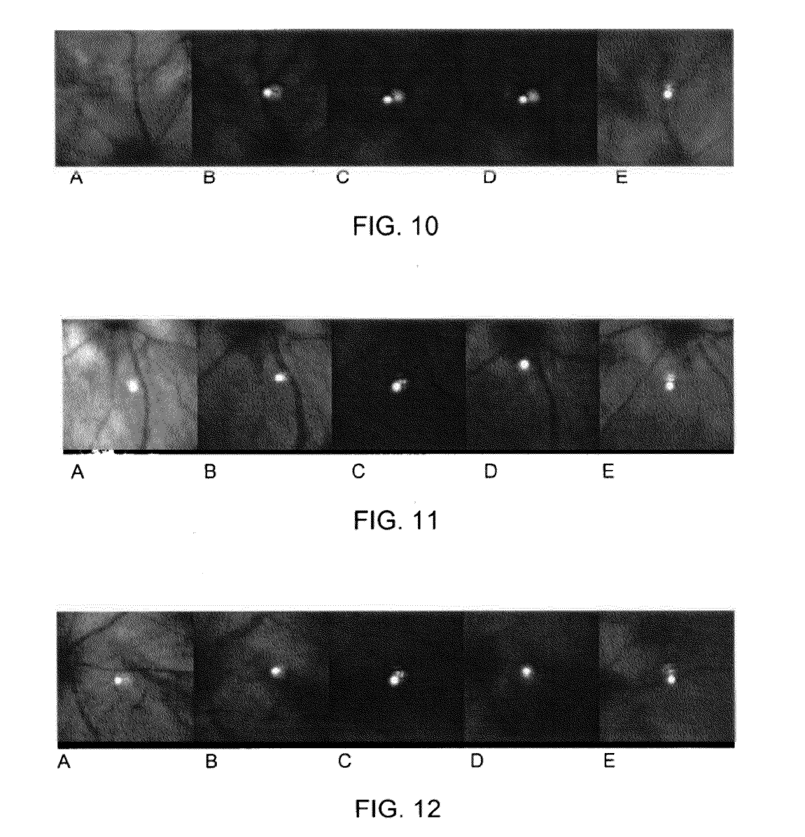

Scanning laser ophthalmoscope (SLO) images (FIGS. 10-15) of retina and digital microscope images of cornea and iris were obtained before the LPS induction and 24, 48, 72, and 96 hours after the LPS injection (and treatment).

### Statistical Analysis

All statistical data were expressed as mean and SEM. Data were analyzed by one-way ANOVA using SigmaStat. Values of p<0.05 were considered significant.

### Wound Infection Animal Study Results

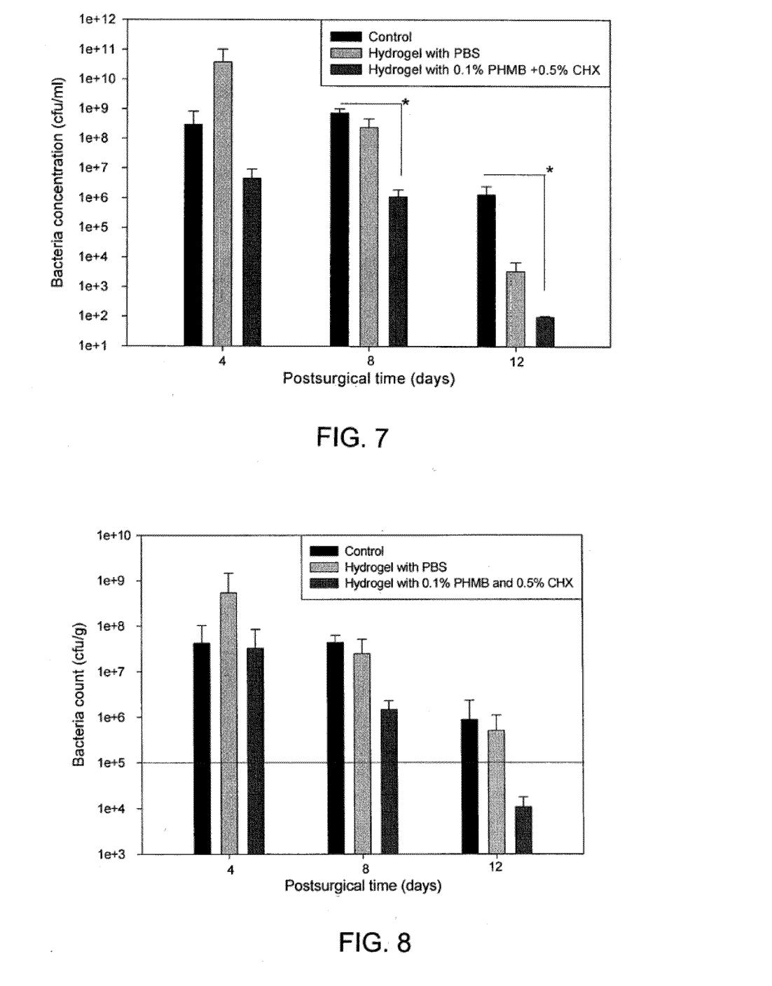

From previous phase studies, it was demonstrated that thermo-responsive hydrogels loaded with 0.1% and 1% PHMB significantly decrease surface bacteria count on day 8 and day 12 after surgery and bacteria inoculation. However, the bacteria count in deep skin samples did not decrease significantly. In this testing, a combination of two antibacterials, 0.1% PHMB and 0.5% chlorhexidine digluconate (CHX), were loaded in thermo-responsive hydrogel and used to treat an infected wound model. As summarized in FIG. 7, the surface bacteria count showed a similar result as described with PHMB alone, where there was a significant decrease in bacteria count on days 8 and 12 compared to the control group. On day 4, while there was an increase in bacteria count for hydrogels with PBS, presumably due to an increase in moisture at the wound site, no bacteria count increase was found for the group with the two disinfectants.

As summarized in FIG. 8, the deep infection evaluation still showed no statistically significant decrease in bacteria concentration compared to the control, mostly due to the high standard deviation from the control group. The bacteria counts for the group treated with 0.1% PHMB and 0.5% CHX, however, were >90% lower than the control group at day 8 and day 12. Also, the group treated with 0.1% PHMB and 0.5% CHX reached lower than 10⁵ cfu/g on day 12, which is considered to be below levels defined as an “infected wound” (Martin, L K et al., 2006).

### DSP-PLGA Nanosphere Loaded Thermo-Responsive Hydrogel Results

The sizes of the DSP-PLGA nanospheres were characterized with dynamic light scattering (DLS), and the average diameter was 190 nm, with a low polydispersity index (PI=0.146). The drug encapsulation efficiency in nanospheres, as determined previously, was 39±4%. The nanospheres were incorporated into thermo-responsive hydrogels at different concentrations, and the swelling ratios tested both at room and body temperature. No significant difference was found in swelling behavior of hydrogels with nanospheres incorporation, as listed in Table 1. Based on the more than 10 times difference in the swelling ratios between room and body temperature, the nanosphere incorporated hydrogels still maintained thermo-responsive behavior. However, the LCST was difficult to determine by spectrophotometry, because the presence of nanospheres interfered with absorbance measurements at room temperature.

TABLE 1
Swelling ratios of hydrogel with varying amount of nanospheres

| | | |
|---|---|---|
| Nanospheres | | Body |
| concentration | Room Temperature | Temperature |

| | | | |
|---|---|---|---|
| 0 | mg/ml | 94.9 ± 35.4 | 7.0 ± 1.9 |
| 2.5 | mg/ml | 90.8 ± 6.6 | 6.6 ± 1.5 |
| 5 | mg/ml | 91.1 ± 7.9 | 6.3 ± 0.8 |
| 10 | mg/ml | 95.0 ± 15.2 | 7.6 ± 3.1 |

### In Vitro Activity of DSP Release Samples

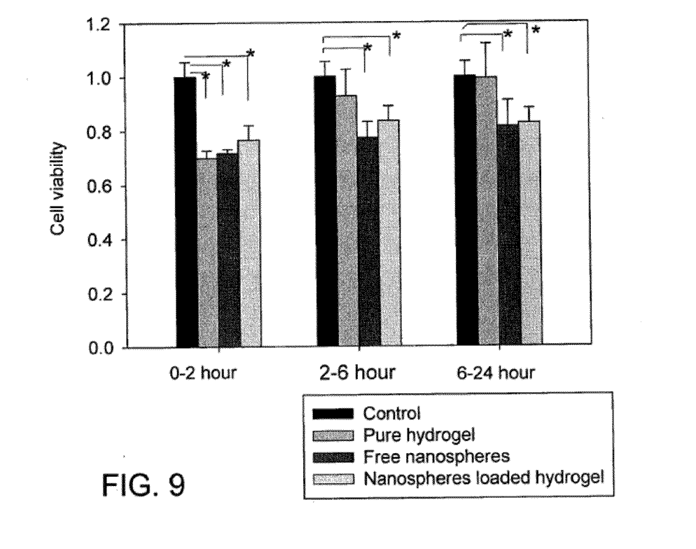

Dexamethasone sodium phosphate (DSP) is a water-soluble inorganic prodrug that is converted into dexamethasone in vivo. DSP has anti-proliferative activity for a variety of cell types, including bone marrow stem cells, white blood cells, fibroblast cells, smooth muscle cells and others. From previous tests, DSP was known to have dose-dependent anti-proliferative activity against mouse fibroblast cells. To investigate the activity of release samples from different drug delivery systems, fibroblast cells proliferation assay was performed again. Release samples from different drug delivery systems were taken out completely at each time and replaced with fresh PBS buffer. From previous tests, it was known that DSP release from pure thermo-responsive hydrogels reached ˜90% release within the first two hours, and the release after that was less than 10%. DSP release from free nanospheres and nanospheres loaded hydrogels maintained sustained release over 24 hours. The drug release data were further confirmed by the activity test for DSP release samples taken at different time intervals, as shown in FIG. 9. The release samples from pure hydrogel only showed significant anti-proliferative activity to reduce cell viability in the first two hours, while the release samples from free nanospheres and nanospheres loaded hydrogels still maintained anti-proliferative activity after two hours and six hours.

### Ocular Application Animal Model

### SLO Images:

Infrared (IR) images were acquired prior to LPS injection and daily up to 96 hours after the LPS injection for three investigated groups. The IR images showed the overall retinal vasculature and the images were used to measure vessel diameters. The progression of uveitis for different treatments is shown in FIGS. 10-12. Overall, a severe impact on the retina was observed by 24 hours and 48 hours post LPS injection for all three conditions, as seen by opaqueness of the vitreous and vasodilation of the vessels. With Treatments 1 and 2, by 96 hours post LPS injection (and 3 applications of treatment) some improvement in the retinal vasculature was observed (FIGS. 10E and 11E) in comparison to non-treatment (FIG. 12E). Though the improvement in the retinal vasculature is somewhat depend on the severity of inflammation. For example, the images from animal shown in FIG. 11 (DSP solution treatment group) had less severe inflammation than DSP-nanosphere-hydrogel animal in FIG. 10.

### Anterior Images:

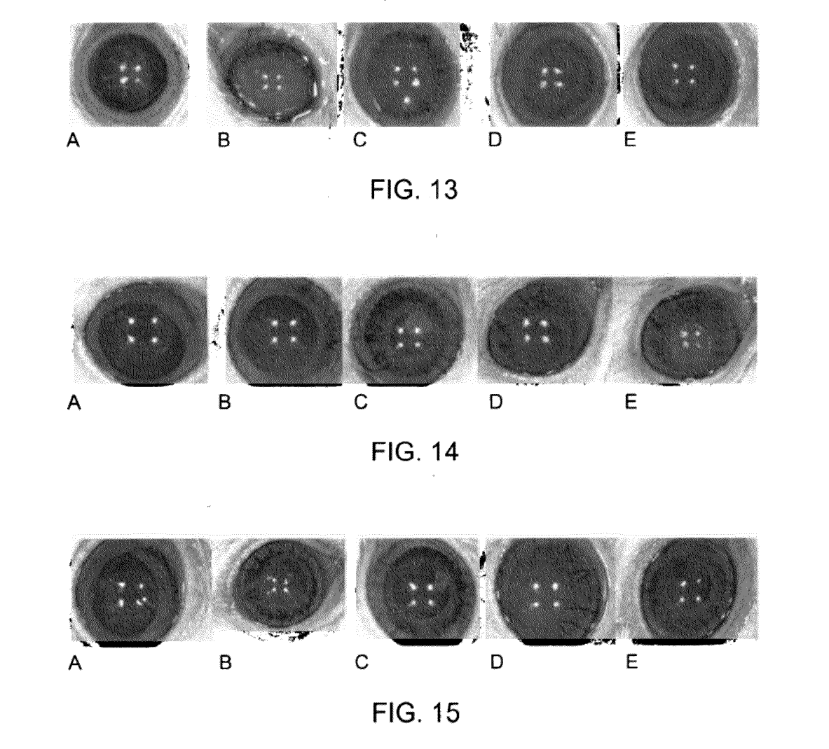

Images of the anterior portion of the eye (cornea, iris) where taken with a digital microscope at the same time points as the SLO images. The purpose of these images was to determine if the LPS-induced inflammation affected both the anterior and posterior portions of the eye in a similar manner since it is possible that the topical treatments might preferentially affect the anterior region. FIG. 13 shows anterior images of the eye of FIG. 10 treated with DSP-nanosphere loaded hydrogel over the course of the experiment. FIG. 14 shows anterior images of the eye of FIG. 11 treated with DSP solution. FIG. 15 shows anterior images of the eye of FIG. 12 that received no treatment. There was a strong correlation between the levels of inflammation seen in the anterior and posterior portions. LPS caused vasodilation of iris vessels which followed a similar trend to that of the retinal vessels.

### Grading of Inflammation:

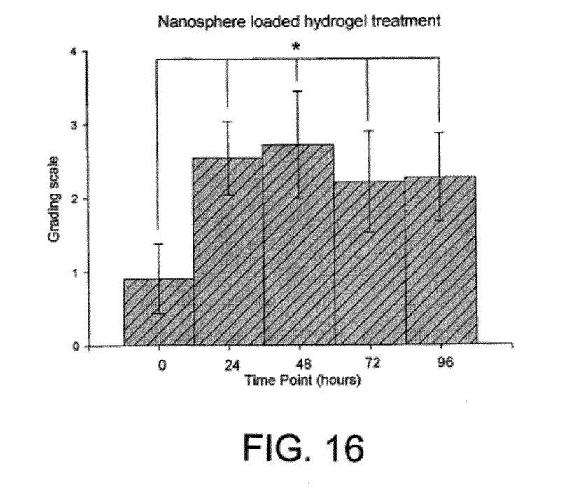

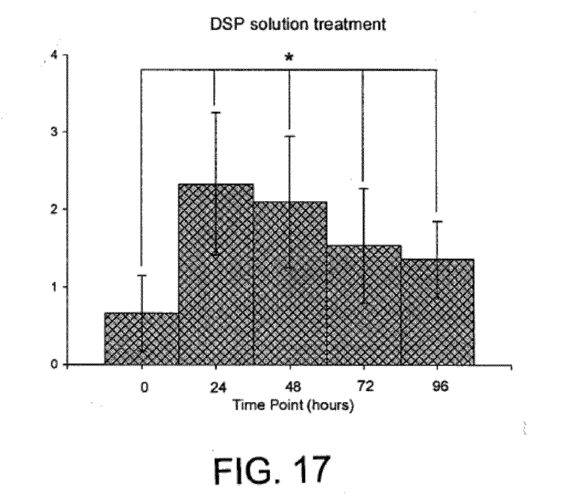

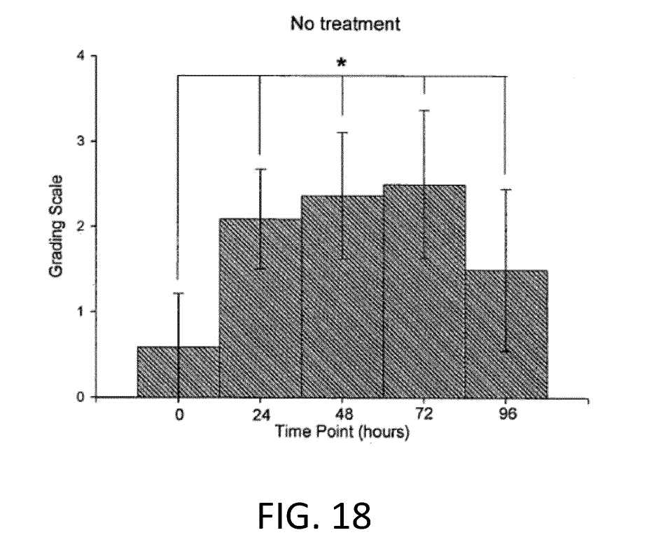

The severity of inflammation was determined by a grading system developed based on a scale from 0 to 4 where grade 0 refers to a clear image and normal vessels while grade 4 represents severe vasodilation and darkened image. One investigator randomized the images from various time points and treatments, while two other investigators graded the images without any knowledge of treatments or time points of the images. Overall, the three treatments studied showed significant inflammation throughout the investigated time frame; however, Treatment 2 seemed to show reduction in inflammation by 72 hours post LPS injection (post 2 applications of treatment) while inflammation in non-treated eyes seemed to continue to increase at 72 hours post LPS. The inflammation for the Treatment 1 group was slightly higher than the LPS-only and Treatment 2 groups at 96 hrs. As seen in FIG. 16, the induced inflammation decreased by 72 and 96 hours (2 and 3 applications of treatment, respectively) in DSP-nanosphere loaded hydrogel treatment group (n=5 eyes). Although there was a decrease in inflammation, the degree of decrease was less than DSP solution topical treatment, as shown in FIG. 17. In Treatment 2 group, by 24 hours post DSP solution treatment, there was a reduction in inflammation that continued up to 96 hours (FIG. 17). In non-treated LPS eyes, as shown in FIG. 18, significant inflammation of retina was observed over the investigated time frame (n=4 eyes). It is important to note that the level of inflammation improvement depend on the initial severity of inflammation at 24 hours post LPS injection.

The severity of inflammation grades indicates that the DSP solution treatments helped reduce inflammation 48 hours after treatment. The mean severity of inflammation decreased for the solution treatment group 48 hours after treatment, while the inflammation in the LPS-only group became more severe at the same time point. Based on the reduction in inflammation seen in the solution treatment group, it seems likely that the DSP was able to penetrate the eye. The current data show that the DSP-nanosphere loaded hydrogel treatment showed minimal decrease in inflammation. The hydrogel treatment group showed a slightly higher severity of inflammation at 96 hours post LPS injection than the LPS-only group. However, the initial severity of LPS inflammation was on average higher in this group compared to the other treatment groups. It seemed that there was a positive correlation of initial severity and outcome of the treatment. It was also possible that the application of the hydrogel treatment could have partially contributed to this, directly or indirectly. Being non-biodegradable, the hydrogel residue could have formed a persistent thin-film over the cornea that would darken the SLO images in a similar way to the clouding of the vitreous and be mistaken for inflammation. The eyes were not rinsed out with buffer after application (in human application, one probably should rinse out the eyes after certain time). It is also possible that drying of the cornea due to anesthesia darkened the images Animals in the hydrogel treatment group were kept immobilized and prevented from blinking for approximately one hour after application of hydrogel under the eyelids to prevent the gel from quickly being blinked out. A small volume of moistening tear drops was applied during this period to try and keep the cornea moist, but the amount of tear drops applied was far less than normal in order to prevent excess tear drop fluid from mixing with the hydrogel. A possible reason for the lack of treatment effects may be the poor residence time of the hydrogels in the eye. After being kept under anesthesia for an hour after treatment, the hydrogel treated animals awoke quickly and began blinking. The blinking motion dislodged the hydrogel from under the eyelid. No hydrogels were observed anywhere on the eye 24 hours after treatment, indicating that the hydrogels were completely expelled from the eye shortly after recovery from anesthesia. The hydrogels were designed to release over 24 hours, so the short residence likely means that significantly less DSP made it into the eye than intended. For clinical application, this may not be a significant problem. The ideal treatment would be to apply the eye drop for overnight treatment and rinse out any remaining hydrogel in the morning. The eye drop will not be applied directly on cornea but deposit under the lid, which was difficult to achieve in small rodent eyes.

### Results Summary

Incorporation of 0.1% PHMB plus 0.5% chlorohexidine digluconate with thermo-responsive hydrogel for wound infection application decreased bacteria count both on the wound surface and in deep skin. PLGA nanospheres incorporated in thermo-responsive hydrogels did not change hydrogel swelling properties. The hydrogels retained their thermo-responsive behavior in the presence of the nanospheres. DSP released from nanospheres-loaded hydrogels retained anti-proliferative activity, and had a more persistent anti-proliferative activity relative to hydrogel release alone. Topically daily applied DSP-PLGA nanospheres encapsulated thermo-responsive hydrogels did not improve LPS-induced inflammation significantly. However, the initial severity of inflammation may play a key factor in governing the success of outcome.

### Example 2

### Ocular Applications

This example demonstrates intravitreal and subconjunctival injections of hydrogels incorporating dexamethasone and/or dexamethasone sodium phosphate.

### Materials

Poly(lactide-co-glycolide) 50:50 (PLGA 50:50; ave. Mw 5,000-15,000), polyvinyl alcohol (PVA; ave. Mw 30,000-70,000), poly(ethylene glycol)diacrylate (PEG-DA; ave. Mn=575), N-isopropylacrylamide (NIPAAm) 97%, n-tert-butylacrylamide (NtBAAm) 97%, N,N,N′,N′-tetramethylethylenediamine (TEMED), ammonium persulfate (APS), dexamethasone ≧97%, dexamethasone 21-phosphate disodium salt ≧98%, lipopolysaccharides (LPS) from Salmonella Typhimurium were obtained from Aldrich-Sigma. Ammonium sulfate was obtained from Acros Organics. Methylene chloride and methanol were obtained from Fisher Scientific in HPLC grade. Acryloyl-lysine or A-lysine (N6-acryloyl lysine) was obtained from CIS Pharma. [1, 2, 4-³H]dexamethasone was obtained from GE Healthcare Life Sciences. Dexamethasone sodium phosphate solution (4 mg/ml, Rx only) was obtained from American Reagent.

### Hydrogel Preparation

Poly (NIPAAm)-PEG hydrogels with A-lysine and NtBAAm were synthesized by free radical polymerization. Specifically, hydrogels with 5% A-lysine and 15% NtBAAm (w/w NIPAAm) were synthesized for ocular application. After hydrogel synthesis, unreacted monomers and initiators were removed by washing in PBS for 5 times, with changing of fresh PBS every 20 minutes. Dexamethasone sodium phosphate at different concentrations (4 mg/ml for American Reagent drug, 10 mg/ml for Sigma-Aldrich drug) was loaded by equilibrating hydrogel in drug solutions overnight for ocular application. After hydrogel synthesis and drug loading, the hydrogels were kept at 4° C. for storage. All hydrogels made for cell and animal experiments were prepared under sterile conditions.

### Preparation of Nanospheres for Dexamethasone Release

Nanospheres were formed at room temperature using an oil in water emulsion (O/W), solvent evaporation technique. Briefly, PLGA (30 mg) and dexamethasone (6 mg) were dissolved in 1 ml of a methylene chloride and methanol mixture (9:1, v./v.). Ten μl of [1, 2, 4-³H]dexamethasone (1 mCi/ml ethanol solution) was then added to the PLGA-dexamethasone oil phase to allow quantification of dexamethasone concentration. The mixture was then added to 20 ml of 2% PVA aqueous solution, followed vortexing for 1 min and then sonication on ice at 55 W for 5 minutes. The resultant emulsion was then stirred at 1250 rpm for 3.5 hours to allow organic solvent evaporation and nanosphere precipitation.

To quantify the size distribution of nanospheres, the suspension was sampled and serially filtered through 0.45 μm, 0.22 μm, and 0.1 μm filters. The drug concentration after each filtration was quantified and calculated within each nanosphere size distribution. Nanospheres with size >100 nm in diameter were harvested by ultrafiltration and centrifugation. The harvested nanospheres were washed twice with DI water to remove excess PVA. The resultant nanosphere suspension was immersed in fresh PBS at 37° C. to initiate release. An equal amount was incubated in 1N NaOH solution at 37° C. to completely degrade the PLGA in order to determine 100% encapsulated. The release samples were taken continuously for 1 day. All samples were quantified for 3H concentration using a scintillation counter. Each experiment was conducted in triplicate.

### Encapsulation and Release of Dexamethasone Sodium Phosphate

PLGA (45 mg) was dissolved in methylene chloride (0.45 ml) followed by the addition of 0.05 ml dexamethasone sodium phosphate methanol solution (100 mg/ml). The clear organic mixture was emulsified into an external aqueous phase (5 ml, 0.25% w/v PVA, with 0.5 N NaCl) with vortexing for 20 seconds. The resulting O/W-emulsion was then immediately poured into 100 ml of 0.25% PVA with 0.5 N NaCl solution and continuously stirred for 3.5 hours at room temperature with a magnetic stirrer. The solid microparticles were separated from external aqueous phase by centrifuging at 4000 rpm for 5 min. The microspheres were then washed twice with 50 ml DI water. The microspheres were suspended in 5 ml DI water after washing.

PLGA nanospheres with dexamethasone sodium phosphate were formed using a protocol modified from PLGA-dexamethasone nanospheres. Briefly, PLGA (30 mg) was dissolved in methylene chloride (0.9 ml) followed by the addition of dexamethasone sodium phosphate (3 mg) dissolved in methanol solution (0.1 ml). The clear organic mixture was then added to 20 ml of 2% PVA aqueous solution with 0.25N ammonium sulfate, followed vortexing for 1 min and then sonication on ice at 55 W for 5 minutes. The resultant emulsion system was then stirred at 1250 rpm for 3.5 hours at room temperature. The nanospheres were harvested by centrifuging followed by 2 times washing with DI water. The nanospheres were suspended in 3 ml DI water after washing.

To test encapsulation efficiency, 1 ml of the PLGA microspheres and nanospheres suspensions were incubated overnight in 1 N NaOH solution at 37° C. to completely degrade PLGA in order to determine the total amount encapsulated in the PLGS. The drug concentration was determined spectrophotometrically at 240 nm. PLGA did not interfere at this wavelength. The encapsulation efficiency was calculated as (actual drug loaded/total drug used)×100%.

To obtain the release profile of dexamethasone sodium phosphate, 1 ml of microspheres or nanospheres suspension was added to 5 ml of fresh PBS (pH=7.4) at 37° C. The samples were taken continuously for the first 24 hours. The drug concentration was measured spectrophotometrically at 240 nm.

### Endotoxin-Induced Uveitis In Vivo Experiment

Adult male Lewis rats (weight 310-350 g) were used as an endotoxin-induced uveitis (EIU) animal model. LPS from Salmonella Typhimurium (Sigma Aldrich, St. Louis, Mo.) was mixed with PBS immediately prior to the injection. The animals were divided as followed:

Control: intravitreal injection of LPS at day 0. ˜5 μl intravitreal injection of control gel (no dexamethasone) at 24 hrs after the LPS induction (day 1);

Treatment control: intravitreal injection of LPS at day 0. ˜5 μl intravitreal injection of dexamethasone (10 mg/ml dose, Sigma) at 24 hrs after the LPS induction;

Treatment 1: intravitreal injection of LPS at day 0. ˜5 μl intravitreal injection of dexamethasone hydrogel (10 mg/ml dose, Sigma) at 24 hrs after the LPS induction; and

Treatment 2: intravitreal injection of LPS at day 0. ˜5 μl subconjunctival injection of dexamethasone hydrogel (10 mg/ml dose, Sigma) at 24 hrs after the LPS induction.

The dexamethasone was loaded by equilibrating 0.1 ml of hydrogel in 2 ml of dexamethasone drug solution. SLO images and blood flow measurement were obtained before the LPS induction, 1, 2, 3, and 6 days after the LPS (and dexametheasone treatment). Furthermore, in one animal, the thermo-hydrogel (control) was applied directly to the cornea (simulate eyedrops).

### Statistical Analysis

All statistical data were expressed as mean and SEM. Data were analyzed by Student's t test using SigmaStat. Values of p<0.05 were considered significant.

### Results

Release Profile of Dexamethasone from PLGA Nanospheres

The release from the low molecular weight PLGA microspheres showed faster release compared to high molecular weight PLGA (85:15, Mw 50 k˜75 k) microspheres, with about 40% release within the first 24 hours. To increase the release rate from the polymer, the size of particles was further decreased to nanoscale by modification of the emulsification protocol. Nanospheres were harvested together with all sizes greater than 100 nm and release carried out at 37° C. in PBS (pH=7.4) in triplicate (n=3). The release profile in FIG. 20 shows that dexamethasone reached about 90% release within the first 20 hours. The faster release from nanospheres than from microspheres is believed due to the smaller diameter.

### Dexamethasone Sodium Phosphate Incorporation to PLGA System

Dexamethasone sodium phosphate is the prodrug of dexamethasone, which is converted to dexamethasone in vivo. Dexamethasone sodium phosphate is more widely used in clinical application as an anti-inflammatory drug due to its high solubility in water (>50 mg/ml).

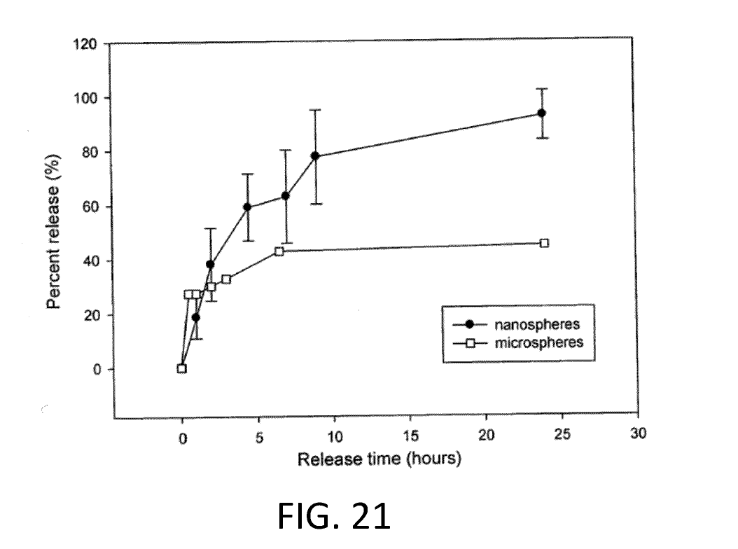

An oil/water (O/W) co-solvent protocol was used to form PLGA microspheres with dexamethasone sodium phosphate. After microsphere formation, digital images were taken and the particles size analyzed. The diameter of PLGA (50:50, Mw 5 k˜15 k) microspheres with dexamethasone sodium phosphate was 14.8±6.0 μm. The release of dexamethasone sodium phosphate at 37° C. was quantified spectrophotometrically at 240 nm. It was found that about 40% of the drug was released within 24 hours, as shown in FIG. 21. Next PLGA nanospheres formation with dexamethasone sodium phosphate was tried with modifications from different protocols. The release was performed the same way as the microspheres with dexamethasone sodium phosphate. It was found that the drug release from nanospheres reached >90% within 24 hours (FIG. 21), which is ideal for overnight application.

### Encapsulation Efficiency

Previous studies experienced low encapsulation efficiency (˜2.5%) for dexamethasone-PLGA microspheres synthesis. A study into the distribution of [1,2,4-³H]dexamethasone determined that most of the drug (˜80%) was lost in the water phase during emulsion, and another 10% was lost during the two washes after emulsion. The rest was lost in some containers, micropipette tips, etc. The encapsulation efficiency is believed to be largely related to the relative partitioning of the solute in oil (O) and water (W) phases. A study of [1,2,4-³H]dexamethasone partitioning in methylene chloride-methanol (O) and 0.2% PVA solution (W) showed that the P=[dex]o/[dex]w=2.93, where O and W have equal volume. However, when microspheres were formed, 40 fold more volume of the water phase than the oil phase was used to reach a desirable particle size. Yet the increase in water volume leads to more distribution of drug into the water phase, which explains the low encapsulation efficiency in the previous studies. This problem can likely be solved by either decreasing the volume of the water phase to allow less drug distribution, or increasing the drug amount to reach saturation in the water phase during emulsion. The latter was tried, because the former causes an increase in particle size, which leads to slower drug release. An increase of dexamethasone in water phase from 1:10 (w/w) of PLGA to 1:2 (w/w) of PLGA increased the partition coefficient from 2.5% to 7.5%.

Another approach to increase partition coefficient is to change the solvent used for the oil and water phases. An increase in PVA concentration from 0.2% to 2% lead to a 3-5 fold increase in partition coefficient of both dexamethasone and dexamethasone sodium phosphate (Table 2). The addition of salt to the water phase was also applied to increase partition coefficient by altering solubility. However, this method was observed to be more effective on dexamethasone sodium phosphate than on dexamethasone Ammonium sulfate appeared to be a more effective salt than sodium chloride (Table 2).

TABLE 2
Partition coefficient of dexamethasone
and dexamethasone sodium phosphate

| | | | P (oil/water) of |
|---|---|---|---|
| | | P (oil/water) of | dexamethasone |
| Oil phase | Water phase | dexamethasone | sodium phosphate |
| Methylene | 0.2% PVA | 2.9 | 0.06 |
| chloride + methanol | | | |
| Methylene | 2% PVA | 12.3 | 0.15 |
| chloride + methanol | | | |
| Methylene | 2% PVA + | 9.6 | 0.26 |
| chloride + methanol | 0.5N NaCl | | |
| Methylene | 2% PVA + | 6.0 | 0.86 |
| chloride + methanol | 0.25N | | |
| | (NH₄)₂SO₄ | | |

The encapsulation efficiencies of dexamethasone and dexamethasone sodium phosphate in microspheres and nanospheres using current protocols are listed in Table 3.

TABLE 3
Encapsulation efficiency of dexamethasone
and dexamethasone sodium phosphate

| | | Encapsulation |
|---|---|---|
| Drug | Particle size | efficiency |
| Dexamethasone | Microspheres | 7.5%* |
| Dexamethasone | Nanospheres | 10%* |
| Dexamethasone | Microspheres | 7.6%^{#} |
| sodium phosphate | | |
| Dexamethasone | Nanospheres | 30%^{#} |
| sodium phosphate | | |

*Determined using 3H-dexamethasone;
^{#}Determined by spectrophotometry at 240 nm

### Ocular Application Animal Model

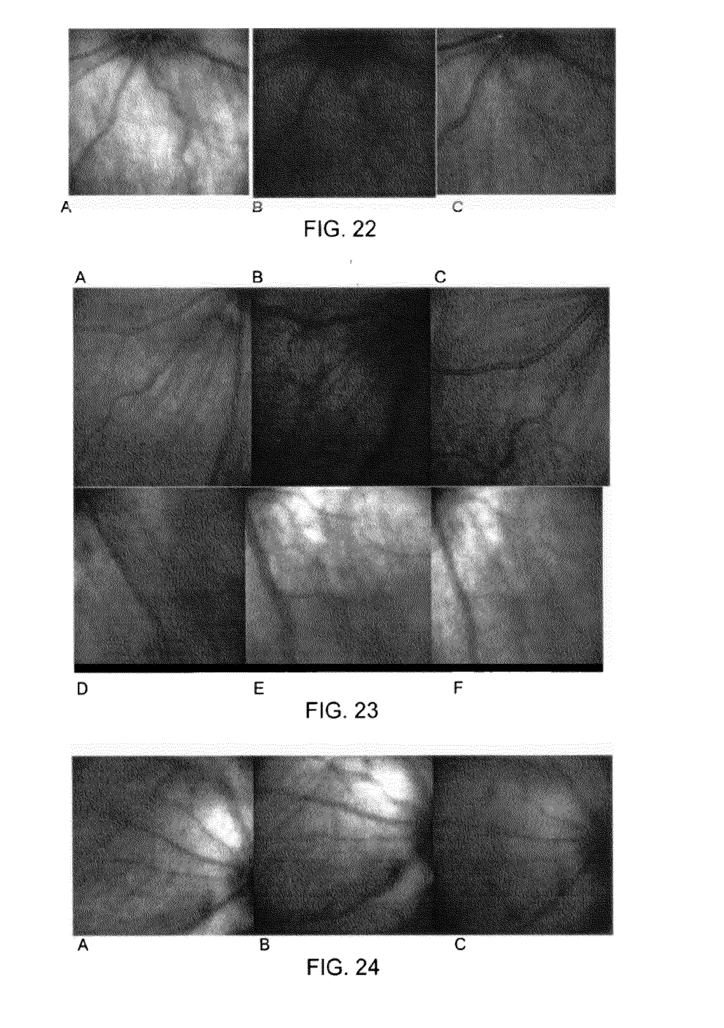

Dexamethasone treatment via thermo-responsive hydrogels was examined FIG. 22 shows the SLO images from the control group. In the control group, after the LPS induction, the control hydrogel (no drug) was injected into the vitreous. SLO images cannot be obtained until 24 hours after the LPS injection due to severe inflammation. At day 2 after the LPS injection (one day after the control hydrogel injection), the images were poor. By day 6, the image quality improved but the measurements were difficult to obtain.

The level of improvement in inflammation was compared by direct injection of dexamethasone and by dexamethasone loaded thermo-responsive hydrogel (n=5 rats), with the resulting images shown in FIG. 23. The LPS injection yielded ˜45% vasodilation of retinal vessels. By 24 hours after the treatment of dexamethasone (by either treatment method), the quality of the images improved though the vessel dilations were present. However, the degree of dilation was reduced to ˜15%. By day 6, the vasotone of retinal vessels returned near normal. Dexamethasone released from the hydrogel had a positive impact on the LPS inflammation. The level of improvement is comparable to the direct treatment of dexamethasone. However, it was noted that dexamethasone from Sigma was less potent than clinical dexamethasone sodium phosphate and the dose concentration was increased (10 mg/ml vs. 4 mg/ml).

The thermo-responsive hydrogel was also subconjunctivally injected and SLO images as shown in FIG. 24 were obtained. There was an improvement with the subconjunctival injection; however, the level of inflammation after the LPS injection was not severe in this animal as measured by the degree of vasodilation (˜12%).

### Summary

Both dexamethasone and dexamethasone sodium phosphate were successfully incorporated into PLGA nanospheres, and their release within 24 hours reached ˜90%. The encapsulation efficiency was above 10%. The intravitreally injected dexamethasone loaded thermo-responsive hydrogels had a positive impact on the inflammation, and the effectiveness of hydrogel treatment was similar to that of direct injection of dexamethasone. The preliminary testing of subconjunctival injection suggested that subconjunctival injection is also a suitable delivery method.

### Example 3

### Cell Adhesion

Thermo-responsive hydrogels were synthesized based on free radical initiated polymerization. A combination of N,N,N′,N′-tetramethylethylenediamine (TEMED) and ammonium persulfate (APS) were used as initiators. Polymerization proceeded at 0° C. for an hour. The incorporation of the monomer A-lysine increased the LCST of the hydrogels because of its hydrophilic nature, which was further adjusted to desirable values by the incorporation of the more hydrophobic monomer N-tert-butylacrylamide (NtBAAm). PEG-DA-575 was used as crosslinker for the hydrogel. The crosslinker density is critical to the hydrogel mechanical property. To make the hydrogel injectable for needles around 27 G, 2 mM PEG-DA was used. The exact composition of thermo-responsive hydrogel synthesis is summarized in Table 4. It should be noticed that the addition of TEMED immediately triggers the hydrogel polymerization, thus should be the last component added to the hydrogel precursor. Also, since the material is highly temperature sensitive, the temperature during the reaction is critical to the final hydrogel property. It is recommended that the reaction be carried out on ice to absorb the heat generated from polymerization and to keep the reaction at a constant temperature.

TABLE 4
Hydrogel composition

| | |
|---|---|
| PEG-DA -575 | 2 mM, 1 μl/ml of total volume |
| NIPAAm | 40 mg/ml of total volume |
| A-Lysine | 5% (w/w) of NIPAAm, |
| | 2 mg/ml of total volume |
| NtBAAm | 20% (w/w) of NIPAAm, |
| | 8 mg/ml of total volume |
| APS | 3 mg/ml of total volume |
| TEMED | 30 μl/ml of total volume |
| PBS (pH 7.4) | Total volume |

### Hydrogel Washing

Initiators and unreacted monomers of the hydrogel exhibit some cytotoxicity and thus should be removed prior to hydrogel application. Residual molecules were extracted by repeated extraction with large volumes of PBS (1 ml of hydrogel to 25 ml of PBS, agitate for 20 min) following polymerization. The pH of the surrounding solution decreased from ˜10 to 7.4 after five extractions. A fibroblast cell culture model was used to investigate the toxicity of hydrogel extracts to identify the number of extractions required for removal of the toxic residues. The MTS assay showed a decrease in viable cells only after exposure to the first and second extraction solution samples. The results demonstrated that the hydrogels synthesized for either application do not exhibit cell toxicity after three extractions. As a conservative standard protocol, five extractions were used.

### Hydrogel Sterilization

Sterilization of hydrogels is necessary for medical application. The hydrogel was sterilized by sterile filtering the precursor and initiator and performing all steps in a laminar flow hood under sterile environment. While this method is realistic for small research experiments, it could be time and labor consuming for massive hydrogel production. Autoclave, gas sterilization and γ-irradiation are the most widely used sterilization process in medical research area. Autoclaving (121° C., 20 min) was first investigated as an alternative method of sterilizing the hydrogels. However, the hydrogels lost their thermo-responsive property after autoclave treatment, possibly due to the breakdown of molecular structure during autoclave sterilization. Ethylene oxide gas sterilization was then investigated for hydrogel sterilization. Wet hydrogels that underwent gas sterilization maintained the original thermo-responsive behavior, with no significant change in LCST or swelling ratio, thus it is recommended to use ethylene oxide gas sterilization for future production of hydrogels for medical applications.

### Testing and Results

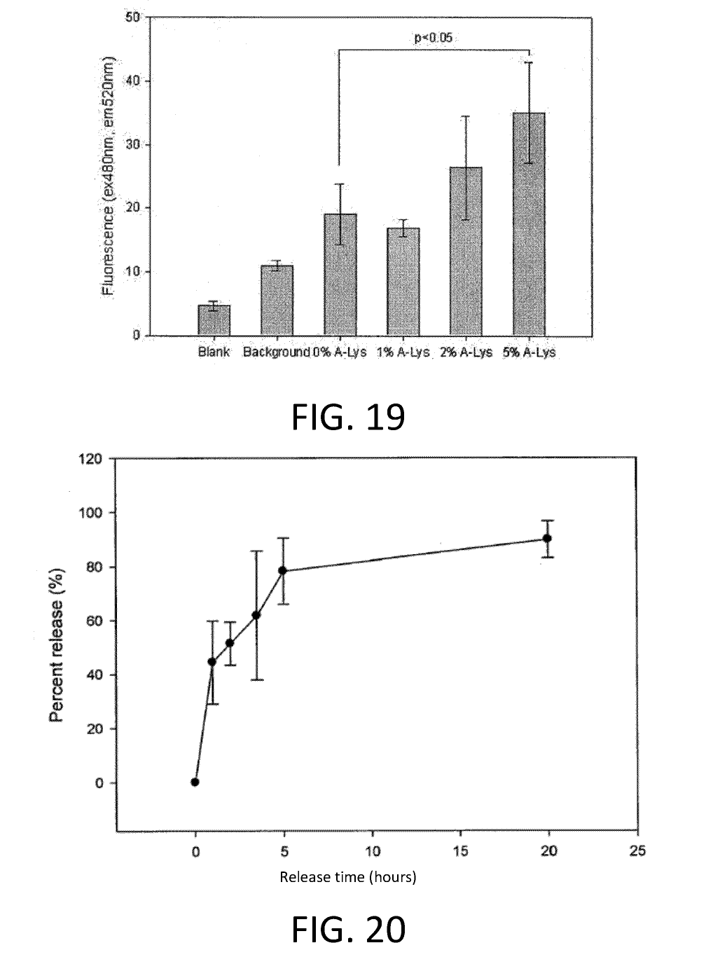

Incorporation of A-lysine into crosslinked PEG-DA gels is expected to improve biocompatibility and cell interactions. Fibroblasts were seeded on crosslinked hydrogels with 5% A-lysine and 20% NtBAAm and gels without A-lysine. Colonies of spread cells were found throughout the surface of the A-lysine containing gels. Cells did not adhere to the hydrogels without A-lysine. Cell adhesion to hydrogels with varying A-lysine concentrations as shown in FIG. 19 was also tested. The quantification was performed using a Pico-Green assay kit to test the DNA concentration on the different hydrogels. The results are summarized in FIG. 19. Only the hydrogel with 5% A-lysine has a significantly (p<0.05) higher DNA concentration compared with pure PNIPAAm-PEG-DA hydrogel (with 0% A-lysine), which was consistent with images of cell adhesion that were taken.

Thus, the invention provides a thermo-responsive hydrogel having improved biological properties, such as having desirably release kinetics and/or cell and tissue interactions, biocompatibility, and/or adhesion. The hydrogel composition can include any of various treatment agents, and is suitable for injectable and topical formulations.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

1. (canceled)

2. The composition of claim 29, further containing a drug composition, a biomolecule, and/or a nanoparticle.

3. (canceled)

4. The composition of claim 29, wherein the amino acid is lysine, tyrosine, serine, cysteine, proline, or combinations or derivatives thereof.

5. (canceled)

6. The composition of claim 29, wherein the crosslinker is a poly(ethylene glycol)diacrylate or a dithiol functionalized molecules.

7. The composition of claim 29, wherein the acrylamide is N-isopropylacrylamide.

8. (canceled)

9. The composition of claim 29, wherein the monomer that alters the lower critical solution temperature of the hydrogel is N-tert-butylacrylamide (NtBAAm).

10. The composition of claim 29, wherein the composition is for wound treatment.

11. (canceled)

12. (canceled)

13. (canceled)

14. The composition of claim 33, wherein the encapsulated drug composition is nanosphere encapsulated dexamethasone.

15. (canceled)

16. The hydrogel composition of claim 2, wherein the thermo-responsive hydrogel has as the acrylamide N-isopropylacrylamide that is crosslinked with poly(ethylene glycol)diacrylate.

17. (canceled)

18. The hydrogel composition of claim 16, wherein the amino acid is lysine, tyrosine, serine, cysteine, proline, or combinations or derivatives thereof.

19. The hydrogel composition of claim 16, wherein the composition is for a wound treatment, and the drug composition comprises an antimicrobial agent.

20. The hydrogel composition of claim 16, wherein the hydrogel composition is for a topical ocular treatment, and the drug composition is encapsulated in a nanosphere.

21-28. (canceled)

29. A thermo-responsive hydrogel composition synthesized by radical polymerization of
(a) an acrylamide and
(b) a monomer containing an amino acid that is linked through its side chain to an acrylic, maleic or phtalic group or derivative,
c) a crosslinker, and
(d) a monomer that alters the lower critical solution temperature of the hydrogel;
wherein the amino acid-containing monomer increases the critical solution temperature of the hydrogel, and the monomer that alters the lower critical solution temperature of the hydrogel adjusts thermo-responsiveness to the desired temperature range from about 32° C. to about 37° C.

30. The composition of claim 29, wherein the crosslinker is polyethylene glycol)diacrylate.

31. The hydrogel composition of claim 2, wherein the composition is for wound treatment, and the drug composition comprises an antimicrobial agent.

32. The hydrogel composition of claim 2, wherein the hydrogel composition is for topical ocular treatment, and the drug composition is encapsulated in a nanosphere.
